(19) United States
(12) Patent Application Publication
(10) Pub. No.: US 2020/0335185 A1
(43) Pub. Date: Oct. 22, 2020

(54) Methods for Educating Patients

(71) Applicant: Patient's Path, LLC, San Antonio, TX (US)

(72) Inventor: Brent Douglas Martin, San Antonio, TX (US)

(73) Assignee: Patient's Path, LLC, San Antonio, TX (US)

(21) Appl. No.: 16/389,900

(22) Filed: Apr. 19, 2019

(51) Int. Cl. G16H 10/40; G16H 10/60; G16H 15/00; G09B 5/06; G16H 10/20

(52) U.S. Cl. CPC G16H 10/40; G16H 10/60; G16H 50/30; G09B 5/06; G16H 10/20; G16H 15/00

(57) ABSTRACT

Methods are disclosed for educating a patient about a medical condition. A healthcare provider (HCP) may collect information about a medical condition of the patient and may transmit the information to a server. The HCP may also take one or more specimens, such as hard or soft tissue, and send them to a medical laboratory. The medical laboratory may retrieve the information from the server, create a medical report based on the information and the one or more specimens, and transmit the medical report to the server. The HCP may retrieve the medical report from the server and deliver it to the patient. Based on the medical report, the HCP or the medical laboratory may recommend one or more educational videos for the patient to view which may educate the patient on the medical condition.

### TECHNICAL FIELD

The present disclosure generally relates to methods for educating a patient about a medical condition of the patient. This may assist the patient in making informed decisions about treatment options for the medical condition.

### BACKGROUND

As background, educating a patient about a medical condition of the patient is a primary goal of healthcare providers. The patient may visit a healthcare professional (HCP) with a medical condition, and the HCP may collect information from the patient about the medical condition and may take one or more specimens from the patient. The HCP then may send the information and specimens to a medical laboratory to be analyzed. The medical laboratory may create a medical report, which is ultimately delivered to the patient by the HCP.

It would be advantageous for the HCP or the medical laboratory to recommend to the patient that the patient view one or more educational videos based on the medical report. Educational videos may be professionally prepared and may include graphs, charts, statistics, and other data which may be more difficult for the HCP to present to the patient in a one-on-one setting. Also, the viewing of the educational videos may allow the HCP to be more efficient with his or her time since a portion of the patient's education can be performed via the educational videos. Furthermore, the educational videos may include information that the HCP may not remember to discuss with the patient, particularly for complex medical conditions.

Furthermore, smaller healthcare providers may not have the benefit of an in-house medical laboratory, and these providers may need to work with a third-party medical laboratory in order to have the information and specimens analyzed. Oftentimes, the health care providers and the medical laboratories use electronic healthcare record (EHR) systems which may not be in communication with each other or may not be compatible with each other. This can make the exchange of information between the healthcare provider and the medical laboratory difficult, possibly requiring human intervention and the attending possibility of human error. Thus, the embodiments shown and described herein allow a patient to be educated about a medical condition of the patient and also allow an HCP and a medical laboratory to effectively exchange information with each other.

As an example of the prior art, U.S. Pat. No. 6,014,630 discloses a system for producing individualized patient educational reports for patients about to receive medical procedures. The reports include data relating to the particular medical procedure, the particular medical facility, and the particular physician. The system includes a database with information regarding: the medical facility where the procedure will be performed, the physician who will perform the procedure, information about the procedure itself, and the medical facility where the procedure will be performed. A user inputs this data manually to the system, which creates a document for the patient that describes what to expect from the procedure. However, this system does not teach a server through which an HCP and a medical laboratory can transfer information. Nor does this system account for the HCP or the medical laboratory selecting educational videos for the patient to view based on the medical report of the patient.

As another example of the prior art, U.S. Patent Application Publication 2018/0247028 discloses a patient engagement and education system having “education assets.” However, this system primarily uses a computer in communication with the patient's electronic healthcare record to automatically assign education assets for a patient to view which are not based on the medical report of the patient. Also, this system does not provide a mechanism for a healthcare professional to communicate to a medical laboratory through a common server.

As yet another example of the prior art, U.S. Patent Application Publication 2018/0286509 teaches a method for educating a patient with education modules. However, these modules are also selected by a controller, and there is no mechanism for a healthcare provider to communicate to a medical laboratory through a common server. Also, the selected education modules are typically based on an upcoming or past surgery and not on a particular medical condition of the patient.

The methods disclosed herein overcome the inherent disadvantages in each of the prior art references by providing a mechanism for an HCP and medical laboratory to efficiently exchange information with each other and for one of them to select one or more educational videos for the patient to view based on the medical report for the patient.

Limitations in the prior art will become apparent to those skilled in the art after reviewing the specification below and the corresponding drawings.

### SUMMARY

In one embodiment, a method for educating a patient about a medical condition of the patient is disclosed. The method comprises: collecting information from the patient about the medical condition of the patient by a healthcare professional (HCP); transmitting the information about the medical condition of the patient to a server by the HCP; retrieving the information about the medical condition of the patient from the server by a medical laboratory; creating a medical report by the medical laboratory, wherein the medical report is based on the information about the medical condition of the patient; transmitting the medical report to the server by the medical laboratory; retrieving the medical report from the server by the HCP; selecting, from an educational video database by the HCP, one or more educational videos for the patient to view based on the medical report, wherein the educational video database comprises a plurality of educational videos; delivering the medical report to the patient by the HCP; and recommending to the patient by the HCP that the patient view the selected one or more educational videos.

In another embodiment, a method for educating a patient about a medical condition of the patient is disclosed. The method comprises: collecting information from the patient about the medical condition of the patient by a healthcare professional (HCP); transmitting the information about the medical condition of the patient to a server by the HCP; retrieving the information about the medical condition of the patient from the server by a medical laboratory; creating a medical report by the medical laboratory, wherein the medical report is based on the information about the medical condition of the patient; selecting, from an educational video database by the medical laboratory, one or more educational videos for the patient to view based on the medical report, wherein the educational video database comprises a plurality of educational videos; transmitting the medical report and a list of the selected one or more educational videos to the server by the medical laboratory; retrieving the medical report and the list of the selected one or more educational videos from the server by the HCP; delivering the medical report to the patient by the HCP; and recommending to the patient by the HCP that the patient view the selected one or more educational videos.

In yet another embodiment, a non-transitory computer-readable storage medium having programmed instructions is disclosed. When the programmed instructions are executed by a processor, they direct the processor to receive information from a healthcare professional (HCP) about a medical condition of a patient; transmit the information about the medical condition of the patient to a medical laboratory; receive a medical report from the medical laboratory, wherein the medical report is based on the information about the medical condition of the patient; and transmit the medical report to the HCP.

### BRIEF DESCRIPTION OF THE DRAWINGS

The embodiments set forth in the drawings are illustrative and exemplary in nature and not intended to limit the inventions defined by the claims. The following detailed description of the illustrative embodiments can be understood when read in conjunction with the following drawings, where like structure is indicated with like reference characters and in which:

FIGS. 1A and 1B depict a flowchart for a method to educate a patient about a medical condition of the patient according to one or more embodiments shown and described herein;

FIG. 2 illustrates a block diagram for a method to educate a patient about a medical condition of the patient according to one or more embodiments shown and described herein;

FIGS. 3A and 3B show a server and an educational video database according to one or more embodiments shown and described herein;

FIG. 4 depicts an educational video database according to one or more embodiments shown and described herein;

FIGS. 5A and 5B show a medical laboratory according to one or more embodiments shown and described herein;

FIG. 6 illustrates information about the medical condition of the patient according to one or more embodiments shown and described herein; and

FIG. 7 depicts a medical report according to one or more embodiments shown and described herein.

### DETAILED DESCRIPTION

The embodiments described herein generally relate to methods for educating a patient about a medical condition of the patient. The methods may include the patient visiting a healthcare professional (HCP) and providing information to the HCP about the medical condition of the patient. The HCP may also take one or more specimens from the patient and send them to a medical laboratory. The HCP may also transmit the information about the medical condition of the patient to a server. The medical laboratory may retrieve the information from the server and create a medical report based on the information and based on an analysis of the one or more specimens. The medical laboratory may then transmit the medical report to the server, which may be retrieved by the HCP. The HCP then may deliver the medical report to the patient. The HCP or the medical laboratory may also select, from an educational video database, one or more educational videos for the patient to view based on the medical report. The educational video database may have a plurality of educational videos which may be designed to help educate patients on various medical conditions. The HCP may further recommend that the patient view the selected one or more educational videos in order to educate the patient regarding the medical condition of the patient.

As used herein, the phrase “healthcare professional” (HCP) is defined to be doctors, dentists, nurses, nurse practitioners, physician assistants, dental assistants, and so forth. Although the HCP may indicate a single individual, it is also intended to signify two or more people acting in concert or one or more people acting under the direction of another. As used herein, “specimen” is defined to be all types of tissue, body fluids, or cells removed from the body of the patient. Specimens may include blood samples, urine samples, microorganism cultures, cytological specimens, hard and soft tissue biopsies, etc. As used herein, the phrase “medical report” is defined to be a pathology report and/or a consultation report. The medical report may include any report issued whether clinical (which includes blood cell counts, coagulation studies, blood glucose levels, bacterial/viral cultures, and urinalysis, etc.) or anatomical (which includes gross and microscopic examination of hard and soft tissue).

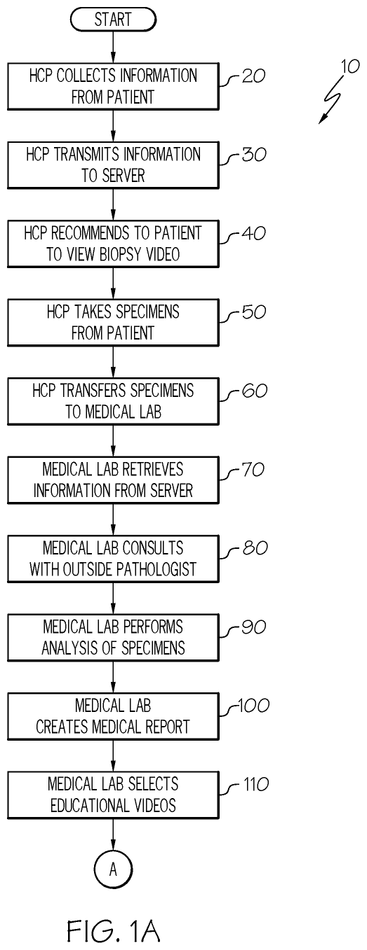

FIGS. 1A and 1B depict a flowchart 10 for educating a patient about a medical condition of the patient. Note that the text inside the box at each step is merely a summary of the action (or actions) that may be taken at that step, and it is not intended to limit the inventions as defined by the claims. At step 20, the HCP collects information from the patient about the medical condition of the patient. Such information may include verbal information from the patient as well as medical images, such as X-rays, photographs, etc. At step 30, the HCP transmits the information about the medical condition of the patient to a server. At step 40, the HCP may recommend to the patient that the patient view one or more educational biopsy videos in order to help the patient decide whether to have a biopsy. At step 50, the HCP may also take one or more specimens from the patient. The one or more specimens may be related to the medical condition of the patient. They may also include one or more biopsies of a pathological entity related to the patient's medical condition. At step 60, the HCP may transfer the one or more specimens to the medical laboratory. This step may include both the sending of the specimens by the HCP as well as the receiving of the specimens by the medical laboratory. At step 70, the medical laboratory may retrieve the information about the medical condition of the patient from the server. In one embodiment, at step 80 the medical laboratory may consult with an outside pathologist regarding the information about the patient and the one or more specimens. Once the one or more specimens have been transferred from the HCP to the medical laboratory, the medical laboratory may perform an analysis of the specimens at step 90. This may include carrying out tests, growing cultures, and so forth on the specimens. At step 100, the medical laboratory may create a medical report based on the information about the medical condition of the patient, (optionally) based on an analysis of the one or more specimens, and/or (optionally) based on the consultation from the outside pathologist.

In one embodiment, at step 110 the medical laboratory may also select, from an educational video database, one or more educational videos for the patient to view based on the medical report. The one or more educational videos may be related to the medical condition of the patient and, when viewed by the patient, may educate the patient on the medical condition of the patient. At step 120, the medical laboratory may transmit the medical report to the server. Also at step 120, if the medical laboratory selects one or more educational videos, the medical laboratory may also transmit a list of the selected one or more educational videos to the server. At step 130, the HCP may retrieve the medical report and the list of the selected one or more educational videos from the server. In another embodiment, at step 140, instead of the medical laboratory selecting the one or more educational videos, the HCP may select, from an educational video database, one or more educational videos for the patient to view based on the medical report. At step 150, the HCP may deliver the medical report to the patient. And at step 160, the HCP may also recommend to the patient that the patient view the selected one or more educational videos in order to educate the patient about the medical condition of the patient. The one or more educational videos may have been selected either by the medical laboratory or the HCP, as discussed above.

At step 170, the patient may view the selected one or more educational videos. This may be done at the HCP's office. In one embodiment, at step 180 the patient may view the selected one or more educational videos under the supervision of the HCP. This may allow the HCP to verify that the patient has actually viewed the educational videos. Also, this may allow the HCP to answer questions from the patient either during the viewing of the videos and/or afterwards. At step 190, the HCP may record in the HCP's electronic healthcare records (EHR) system that the patient has actually viewed the selected one or more educational videos.

Note that all steps of the flowchart 10 do not necessarily need to be performed in the exact order shown in FIGS. 1A and 1B. For example, steps 30 and 60 do not need to be performed in the order shown. That is, the HCP may transfer the one or more specimens to the medical laboratory before transmitting the information about the medical condition of the patient to the server. Thus, it should be apparent to those skilled in the art which steps should be performed in the order shown and which steps do not need to be performed in the order shown.

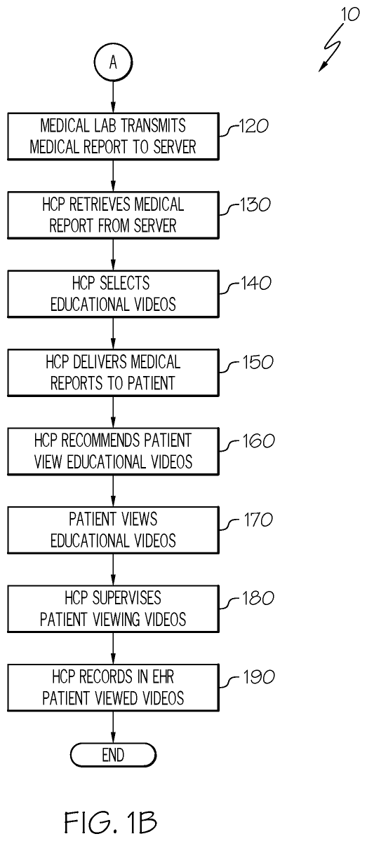

FIG. 2 shows a block diagram 200 of a method for educating a patient on a medical condition of the patient. The patient 210 may have a medical condition 210M and may visit an HCP 220 in order to seek help and advice on alleviating the medical condition 210M. The HCP 220 may collect information from the patient 210 about the medical condition 210M of the patient 210. Such information may include verbal information from the patient 210 such as when the medical condition began, details of any symptoms, the location and severity of pain, etc. The HCP 220 may also collect scientific information from the patient 210 such as medical images, photographs, measurements of size, shape, and surface changes of a pathological entity related to the medical condition of the patient. A pathological entity may comprise hard or soft tissue and may include but are not limited to benign or malignant entities such as cysts or tumors as well as immune-mediated conditions, whether bacterial, viral, or fungal-related. The medical images may include any type of image of the patient, including but not limited to X-rays, CT scans, CBCT scans, MRI scans, PET scans, and so forth. All of the information, verbal and scientific, may be collected by the HCP 220 from the patient 210 at step 20.

The HCP 220 may also take one or more specimens from the patient 210 at step 50, wherein the one or more specimens may be related to the medical condition 210M of the patient 210. As defined above, such specimens may include but are not limited to all types of tissue, body fluids, or cells removed from the body of the patient. The specimens may also include one or more biopsies of a pathological entity related to the medical condition 210M of the patient 210. The specimens may be related to the medical condition of the patient in such a manner that their analysis may directly indicate the cause of the patient's condition. Also, some specimens may be taken in order to rule out a particular diagnosis; that is, the absence of finding a particular organism or compound may assist the medical laboratory in creating the medical report. These specimens may be taken from the patient 210 by the HCP 220 at step 50. After the one or more specimens have been taken, the HCP 220 may transfer them to a medical laboratory 240 for analysis at step 60. The one or more specimens may be transferred to the medical laboratory 240 by courier, postal service, shipping company, or any other suitable means.

The HCP 220, having collected from the patient 210 information related to the medical condition 210M of the patient 210, may transmit this information to a server 230 at step 30. This information may be transmitted electronically through the internet or other suitable means. The server 230 may comprise a computer, website, or any other suitable means which may receive and store the information about the medical condition 210M of the patient 210. The HCP 220 may need to log onto the server 230 using a username and password, using a personal computer, tablet, smart phone, etc. The link between the HCP 220 and the server 230 may be encrypted so that the information transmitted to the server 230 is secure. Standard encryption techniques may be used, as is known in the art, including but not limited to SSA, AES-128, AES-256, etc. The server 230 may comprise a processor and a non-transitory, computer-readable storage medium 230M such as a hard drive, flash memory, and so forth. The storage medium 230M may have programmed instructions which, when executed by the processor, direct the processor to receive the information from the HCP 220 at step 30. As discussed herein, the programmed instructions may also direct the processor to transmit the information to the medical laboratory 240 at step 60, receive a medical report (and, optionally, a list of selected educational videos) from the medical laboratory 240 at step 120, and transmit the medical report (and, optionally, the list of selected educational videos) to the HCP 220 at step 130.

Continuing to refer to FIG. 2, the medical laboratory 240 may retrieve the information about the medical condition 210M of the patient 210 at step 70. As with the HCP 220, the medical laboratory 240 may need to log onto the server 230 using a username and password in order to facilitate a secure link to the server 230. Furthermore, the medical laboratory 240 may use a personal computer, tablet, or smart phone in order to retrieve the information from server 230. The server 230 may also be capable of notifying the HCP 220 or the medical laboratory 240 that the information about the medical condition 210M of the patient 210 and/or the medical report is ready to be retrieved. For example, the server 230 may be capable of sending a text or an email to the HCP 220 and/or medical laboratory 240 indicating that a particular document has been transmitting to the server 230, and that it is now ready to be retrieved.

Once the medical laboratory 240 has retrieved the information about the medical condition 210M of the patient 210 from the server 230 and has received the one or more specimens from the HCP 220, the medical laboratory 240 may create a medical report based on this information and an analysis of the one or more specimens. Creating the medical report may involve analyzing the one or more specimens and taking into account the information about the medical condition 201M of the patient 220. For example, the medical laboratory 240 may culture the one or more specimens in order to determine what kind of bacteria and/or viruses may be present in the specimens.

In one embodiment, the medical laboratory 240 may have sufficient resources to create the medical report. That is, the medical laboratory 240 may be capable of creating the medical report without any outside assistance. In another embodiment, the medical laboratory 240 may want to get the opinion of an outside pathologist 330, that is, a pathologist that is not part of the medical laboratory. This may be done, for example, if the nature of the patient's condition may be unusual or the medical laboratory 240 may want to confirm a particular diagnosis. In such cases, the medical laboratory 240 may request a consultation from a pathologist outside of the medical laboratory 240 at step 80. In this case, the medical report may be further based on the consultation from the outside pathologist 330.

The medical report created by the medical laboratory 240 may include possible causes of the medical condition 210M of the patient 210. In addition, the medical report may include one or more potential strategies in order to successfully treat the medical condition 210M. That is, the medical report may be structured in order to provide the HCP 220 with sufficient information to advise the patient 210 on the cause of the medical condition 210M as well as treatment options to alleviate or cure the medical condition 210M.

After the medical laboratory 240 has created the medical report, in one embodiment, the medical laboratory 240 may also select one or more educational videos, based on the medical report, which may help educate the patient 210 about his or her medical condition 210M. The educational videos may be stored in an educational video database on the server 230 or may be stored in an educational video database that is accessible to both the medical laboratory 240 as well as the HCP 220. If the medical laboratory 240 selects the one or more educational videos, the medical laboratory 240 may transmit the medical report and a list the selected one or more educational videos to the server 230 at step 120. Both the medical report and the list of the selected one or more educational videos may be transmitted at the same time to the server 230, but they may also be transmitted separately. The server 230 may be capable of storing the medical report and the list of the selected one or more educational videos.

Continuing to refer to FIG. 2, the HCP 220 may retrieve the medical report from the server 230 at step 130. If the medical laboratory 240 also selected one or more educational videos for the patient 210 to view, the HCP 220 may also retrieve the list of the selected one or more educational videos from the server 230 at step 130. In another embodiment, the HCP 220 selects one or more educational videos for the patient 210 to view instead of the medical laboratory 240. In this embodiment, the HCP may retrieve just the medical report from the server 230 at step 130.

At step 150, the HCP 220 delivers the medical report to the patient 210. This is preferably done in person, but it may also be done by phone or video conference. At this step 150, the HCP 220 may discuss with the patient 210 the contents of the medical report, including possible diagnoses and potential treatments for the patient's medical condition. At step 160, the HCP 220 recommends to the patient 210 that the patient 210 view the selected one or more educational videos, which were either selected by the HCP 220 or the medical laboratory 240, as discussed above. The selected one or more educational videos may help educate the patient 210 on the medical condition 210M of the patient 210. They may also help the patient 210 decide on which course of treatment to follow, in cases where there are multiple treatment options.

Continuing to refer to FIG. 2, the HCP 220 may use an electronic healthcare record (EHR) system 220E which may permit the HCP 220 to electronically store and retrieve health records for patients of the HCP 220. Likewise, the medical laboratory 240 may also use an EHR system 240E which may permit the medical laboratory 240 to electronically store and retrieve health records for its patients. Both the HCP 220 and the medical laboratory 240 may be able to access their respective EHR systems 220E, 240E via a personal computer, tablet, smartphone, or any such device as is known in the art. In one embodiment, the EHR systems 220E, 240E of the HCP 220 and medical laboratory 240 are either not in communication with each other or are not compatible with each other. That is, the EHR systems 220E, 240E may be from different vendors and/or may store the healthcare records in a different format. In this case, the server 230 may allow the HCP 220 and medical laboratory 240 to exchange information (i.e., information about the medical condition 210M of the patient 210 and the medical report) with each other effectively and efficiently, even though their respective EHR systems 220E, 240E may not be able to do so.

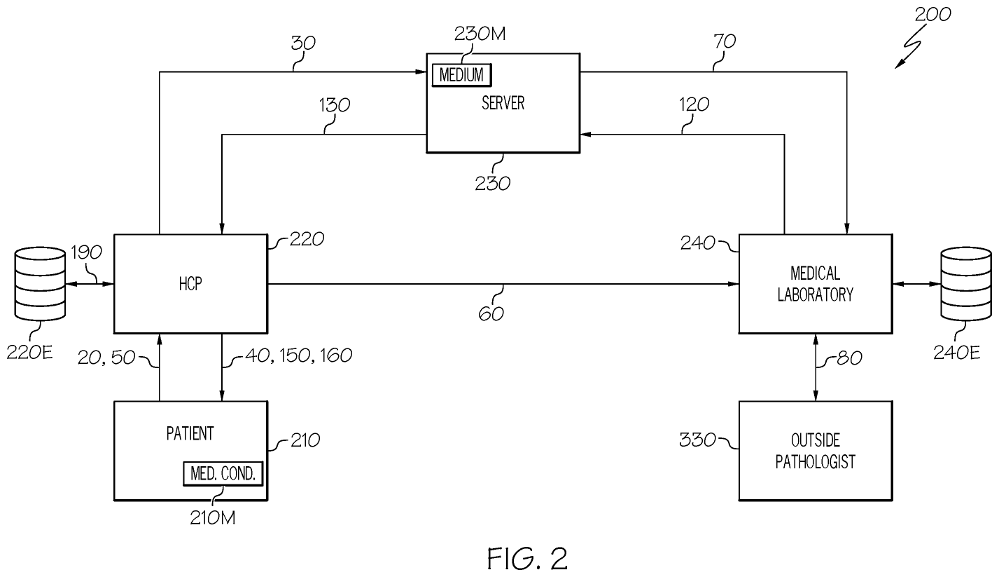

FIGS. 3A and 3B show two possible embodiments of how and where the one or more educational videos are stored in an educational video database (EVDB) 300. In FIG. 3A, the educational videos may be stored in an educational video database 300 directly on the server 230. This is the preferred embodiment since the HCP and the medical laboratory may have direct access to the server 230. FIG. 3B shows an alternative embodiment in which the educational video database 300 is stored on a secondary server 310. In this embodiment, the server 230 and the secondary server 310 may be electrically coupled such that the server 230 may be able to retrieve the educational videos from the educational video database 300 on the secondary server 310. It is contemplated that the educational video database 300 may be structured in any suitable manner such that the HCP and the medical laboratory may have access to the educational video database 300 and the associated educational videos.

As discussed above, the server 230 may comprise a processor and a non-transitory, computer-readable storage medium 230M such as a hard drive, flash memory, and so forth. The storage medium 230M may have programmed instructions which, when executed by the processor, direct the server 230 to receive the information from the HCP and to allow medical laboratory to retrieve the information from the server 230. Likewise, the storage medium 230M may have programmed instructions which, when executed by the processor, direct the server 230 to receive the medical report from the medical laboratory and to allow the HCP to retrieve the medical report from the server 230.

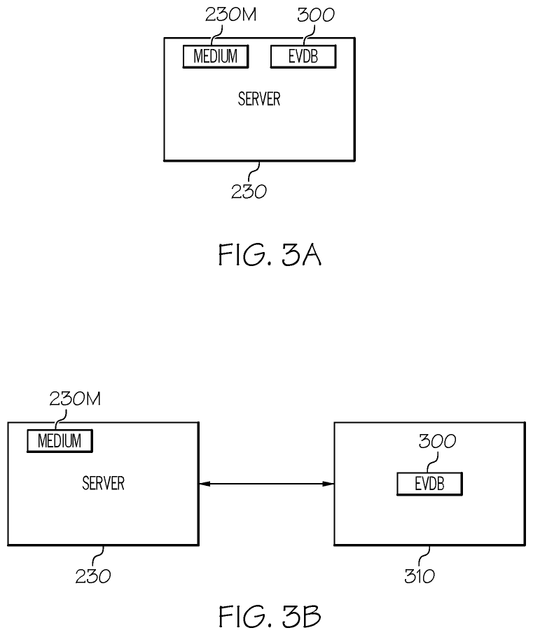

FIG. 4 depicts an educational video database 300 according to one or more embodiments shown and described herein. The educational video database 300 may comprise a plurality of educational videos 300A which, when viewed by the patient, may allow the patient to be educated about the medical condition of the patient. The videos may comprise an introductory section acknowledging the diagnosis that has been rendered. The video may explain what the diagnosis is, for example the tissue from which the entity arose, the causes, effects, prognosis, expectations, follow-up interval if any, and conclusion which would include other videos that might be applicable. The one or more educational videos may be viewed by the patient at the office of the HCP. In one embodiment, the patient may view the videos under the supervision of the HCP so that the patient may ask questions to the HCP during the viewing. This may also allow the HCP to confirm that the patient has actually viewed the one or more educational videos. In this case, the HCP may record this information in an electronic healthcare record (EHR) system of the HCP.

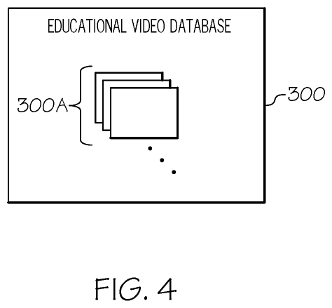

FIGS. 5A and 5B show embodiments in which a pathologist may be used to render an opinion with respect to one or more specimens taken from the patient. In FIG. 5A, the pathologist 320 may work directly for the medical laboratory 240 and may render an opinion on the one or more specimens within the context of the medical laboratory creating the medical report. In this case, the pathologist may be considered an in-house pathologist 320. FIG. 5B shows an alternative embodiment in which the medical laboratory 240 may need to request a consultation opinion at step 80 from an outside pathologist 330, that is, a pathologist working outside the medical laboratory 240. This may be an extra step the medical laboratory 240 may have to take in cases where one or more specimens have been taken from the patient, and the medical laboratory 240 needs to get the opinion of an outside pathologist 330.

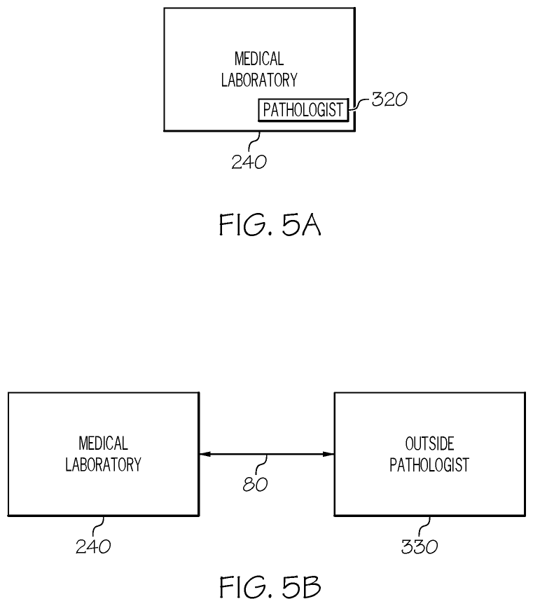

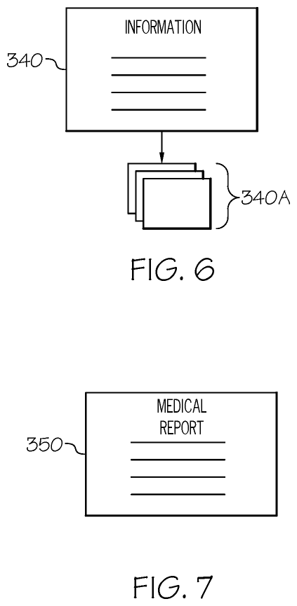

FIGS. 6 and 7 illustrate information 340 about the medical condition of the patient and the medical report 350, respectively. As discussed previously, the information 340 may comprise various types of information 340A, include both verbal and scientific information from the patient. In addition, the information 340 may include some medical history about the patient. The medical report 350 may comprise a consultation report or a pathology report. Both the information 340 and the medical report 350 may be formatted in any suitable format. There may be an advantage in formatting both the information 340 and the medical report 350 in a standardized format that is common in the electronic healthcare records (EHR) business. For example, both the information 340 and the medical report 350 may be formatted in the FHIR® (Fast Healthcare Interoperability Resources) format promulgated by the Health Level Seven International (HL7) health-care standards organization. It is anticipated that other types of formats, including those yet to be created, may be used as well.

While particular embodiments and aspects of the present invention have been illustrated and described herein, various other changes and modifications may be made without departing from the spirit and scope of the invention. Moreover, although various inventive aspects have been described herein, such aspects need not be utilized in combination. It is therefore intended that the appended claims cover all such changes and modifications that are within the scope of this invention.

What is claimed is:

1. A method for educating a patient about a medical condition of the patient, the method comprising:
collecting information from the patient about the medical condition of the patient by a healthcare professional (HCP);
transmitting the information about the medical condition of the patient to a server by the HCP;
retrieving the information about the medical condition of the patient from the server by a medical laboratory;
creating a medical report by the medical laboratory, wherein the medical report is based on the information about the medical condition of the patient;
transmitting the medical report to the server by the medical laboratory;
retrieving the medical report from the server by the HCP;
selecting, from an educational video database by the HCP, one or more educational videos for the patient to view based on the medical report, wherein the educational video database comprises a plurality of educational videos;
delivering the medical report to the patient by the HCP; and
recommending to the patient by the HCP that the patient view the selected one or more educational videos.

2. The method of claim 1, wherein the information about the medical condition of the patient comprises information associated with a pathological entity related to the medical condition of the patient, including at least one of the following: a medical image of the pathological entity; a photograph of the pathological entity; and a measurement of size, color, shape, and surface changes of the pathological entity.

3. The method of claim 1, wherein the information about the patient and the medical report are formatted in FHIR format.

4. The method of claim 1, wherein the HCP uses a first electronic healthcare records (EHR) system and the medical laboratory uses a second EHR system, and the first EHR system and second EHR system are either not in communication with each other or are not compatible with each other.

5. The method of claim 1, wherein the educational video database is stored on the server.

6. The method of claim 1, wherein the method further comprises viewing the selected one or more educational videos by the patient.

7. The method of claim 1, wherein the method further comprises viewing of the selected one or more educational videos by the patient under supervision of the HCP.

8. The method of claim 7, wherein the method further comprises recording, in an electronic healthcare records (EHR) system of the HCP, that the selected one or more educational videos was viewed by the patient.

9. The method of claim 1, wherein the method further comprises recommending to the patient by the HCP that the patient view one or more educational biopsy videos for educating the patient on advantages and disadvantages of having a biopsy.

10. The method of claim 1, wherein the method further comprises:
taking one or more specimens from the patient by the HCP, wherein the one or more specimens are related to the medical condition of the patient;
transferring the one or more specimens to the medical laboratory by the HCP;
performing an analysis of the one or more specimens by the medical laboratory; and
creating the medical report by the medical laboratory is further based on the analysis of the one or more specimens.

11. The method of claim 10, wherein the one or more specimens comprise at least one biopsy.

12. The method of claim 10, wherein the method further comprises getting a consultation by the medical laboratory from an outside pathologist for at least one of the one or more specimens, and creating the medical report by the medical laboratory is further based on the consultation from the outside pathologist.

13. A method for educating a patient about a medical condition of the patient, the method comprising:
collecting information from the patient about the medical condition of the patient by a healthcare professional (HCP);
transmitting the information about the medical condition of the patient to a server by the HCP;
retrieving the information about the medical condition of the patient from the server by a medical laboratory;
creating a medical report by the medical laboratory, wherein the medical report is based on the information about the medical condition of the patient;
selecting, from an educational video database by the medical laboratory, one or more educational videos for the patient to view based on the medical report, wherein the educational video database comprises a plurality of educational videos;
transmitting the medical report and a list of the selected one or more educational videos to the server by the medical laboratory;
retrieving the medical report and the list of the selected one or more educational videos from the server by the HCP;
delivering the medical report to the patient by the HCP; and
recommending to the patient by the HCP that the patient view the selected one or more educational videos.

14. The method of claim 13, wherein the information about the medical condition of the patient comprises information associated with a pathological entity related to the medical condition of the patient, including at least one of the following: a medical image of the pathological entity; a photograph of the pathological entity; and a measurement of size, color, shape, and surface changes of the pathological entity.

15. The method of claim 13, wherein the information about the patient and the medical report are formatted in FHIR format.

16. The method of claim 13, wherein the HCP uses a first electronic healthcare records (EHR) system and the medical laboratory uses a second EHR system, and the first EHR system and second EHR system are either not in communication with each other or are not compatible with each other.

17. The method of claim 13, wherein the educational video database is stored on the server.

18. The method of claim 13, wherein the method further comprises viewing the selected one or more educational videos by the patient.

19. The method of claim 13, wherein the method further comprises viewing of the selected one or more educational videos by the patient under supervision of the HCP.

20. The method of claim 19, wherein the method further comprises recording, in an electronic healthcare records (EHR) system of the HCP, that the selected one or more educational videos was viewed by the patient.

21. The method of claim 13, wherein the method further comprises recommending to the patient by the HCP that the patient view one or more educational biopsy videos for educating the patient on advantages and disadvantages of having a biopsy.

22. The method of claim 13, wherein the method further comprises:
taking one or more specimens from the patient by the HCP, wherein the one or more specimens are related to the medical condition of the patient;
transferring the one or more specimens to the medical laboratory by the HCP;
performing an analysis of the one or more specimens by the medical laboratory; and
creating the medical report by the medical laboratory is further based on the analysis of the one or more specimens.

23. The method of claim 22, wherein the one or more specimens comprise at least one biopsy.

24. The method of claim 22, wherein the method further comprises getting a consultation by the medical laboratory from an outside pathologist for at least one of the one or more specimens, and creating the medical report by the medical laboratory is further based on the consultation from the outside pathologist.

25. A non-transitory computer-readable storage medium having programmed instructions which, when executed by a processor, direct the processor to:
receive information from a healthcare professional (HCP) about a medical condition of a patient;
transmit the information about the medical condition of the patient to a medical laboratory;
receive a medical report from the medical laboratory, wherein the medical report is based on the information about the medical condition of the patient; and
transmit the medical report to the HCP.
